(19) United States
(12) Patent Application Publication
(10) Pub. No.: US 2011/0070624 A1
(43) Pub. Date: Mar. 24, 2011

(54) MICROWAVE RESONANT ABSORPTION METHOD AND DEVICE FOR VIRUSES INACTIVATION

(75) Inventors: Chi-Kuang Sun, Taipei (TW); Tzu-Ming Liu, Keelung (TW)

(73) Assignee: NATIONAL TAIWAN UNIVERSITY, Taipei City (TW)

(21) Appl. No.: 12/562,591

(22) Filed: Sep. 18, 2009

(51) Int. Cl. C12N 7/04; C12M 1/00

(52) U.S. Cl. 435/236; 435/283.1

(57) ABSTRACT

Virus inactivation is performed with a specific microwave frequency to induce a collective vibration of virus through microwave resonant absorption (MRA).

### FIELD OF THE INVENTION

The present invention relates to a non-thermal physical method to inactivate the viruses through microwave resonant absorption. The present invention also relates to a device to inactivate the viruses through microwave resonant absorption

### BACKGROUND OF THE INVENTION

The amino acids of viral capsids or envelopes have complex charge distributions on the surface of viruses. These charges will attract counter ions to the surface and forms an electric double layer. Such core-shell charge separation paves the road for the dipolar coupling between electromagnetic waves and confined acoustic vibrations. The microwave resonant absorption (MRA) processes will thus occur in viruses when the confined acoustic vibration moves the charges and changes their dipole moments. (T.-M. Liu et al. Microwave resonant absorption of viruses through dipolar coupling with confined acoustic vibrations. Appl. Phys. Lett. 94, 043902 (2009)). For spherical viruses, such a vibrational mode is the spheroidal (SPH) mode with an angular momentum l=1. (Lamb, H. On the vibrations of an elastic sphere. Proc. London Math. Soc. 13, 189-212 (1882), Duval, E. Far-infrared and Raman vibrational transitions of a solid sphere: Selection rules. Phys. Rev. B 46, 5795-5797 (1992)). Both the theoretically calculated [SPH, l=1, n=0] and [SPH, l=1, n=1] dipolar modes (See FIG. 1) have relative displacement between the core (indicated by light-colored arrows) and shell (indicated by dark-colored arrows) (Murray, D. B. et al. Far-infrared absorption by acoustic phonons in Titanium dioxide nanopowders. J. Nanoelectron. Optoelectron. 1, 92-98 (2006)), which should induce MRA through the core-shell charge structure of a nanoparticle, including viruses. By assuming a virus as a homogeneous sphere with elastic parameters close to the Satellite Tobacco Mosaic Virus crystal, (Stephanidis, B., Adichtchev, S., Gouet, P., McPherson, A. & Mermet, A. Elastic properties of viruses. Biophys. J. 93, 1354-1359 (2007)) one can estimate the frequency of the [SPH, l=1, n=0] mode for a 30 nm virus to be around 40 GHz. The frequencies of the dipolar modes are determined by the longitudinal sound velocity V_{L}, transverse sound velocity V_{T}, and the radius of viruses R. (Lamb, H. On the vibrations of an elastic sphere. Proc. London Math. Soc. 13, 189-212 (1882)) Assuming V_{L}/V_{T} ˜2 for viruses, the eigen frequencies are expected to be proportional to V_{L}/R For most viruses, their sizes range from 10 nm to 300 nm and their V_{L} doesn't change too much, thus making the frequency of the dipolar modes falls in the microwave range.

U.S. Pat. No. 6,268,200 discloses biotherapeutic virus attenuation using variable frequency microwave energy. The range of microwave frequencies has a central frequency selected to break apart helix strands of a nucleic acid of the virus, to modify a capsid enclosing a nucleic acid of the virus, or to selectively couple to water molecules inside the capsid without harming the biotherapeutic or the container.

### BRIEF DESCRIPTION OF DRAWINGS

FIG. 1 Displacement of the [SPH, l=1, n=0] and [SPH, l=1, n=1] dipolar modes in a free homogeneous sphere. The light-colored arrows and dark-colored arrows indicate the local displacement of the core and shell of a nanosphere, respectively.

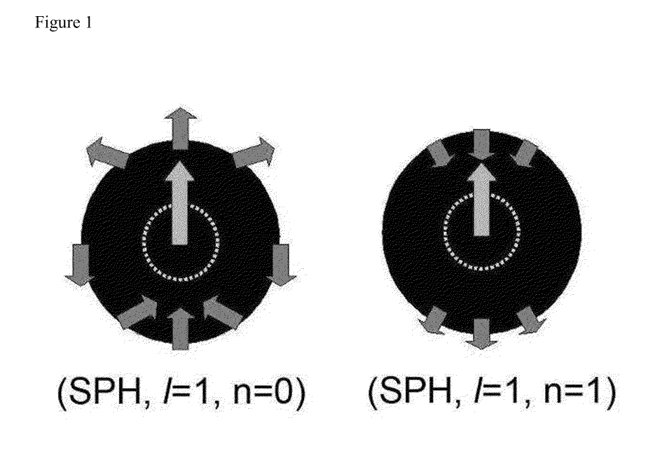

FIG. 2 Schematic diagram of the microwave virus inactivator. It is composed of a YIG oscillator, an active doubler, a power amplifier, an isolator, and a horn antenna. The 150 mm Petri dish is an example of container, containing viruses to be inactivated. Microwave absorbers are used to prevent radiation pollution of microwaves.

FIG. 3 Microwave spectrum of MTT titer of EV71. The dashed line represents the control (Placing viruses beneath the horn antenna without microwave illumination).

### SUMMARY OF THE INVENTION

The present invention provides a non-thermal method of inactivating the virus through microwave resonant absorption.

The present invention also provides a device of inactivating the virus established through the virus inactivation method.

### DETAILED DESCRIPTION OF THE INVENTION

“Inactivation” is the destruction of biological activity, as of a virus, by the action of heat, vibration, chemical or other agent.

“A collective vibration of a virus” is a stationary wave vibration of whole virus structure.

“Stiffness constant” is any one of the coefficients of the relations in the generalized Hooke's law used to express stress components as linear functions of the strain components.

“Bulk modulus (K)” is a substance's resistance to uniform compression, defined as the pressure increase needed to cause a given relative decrease in volume.

“Poisson's ratio (v)” is a ratio, when a sample object is stretched, of the contraction or transverse strain (perpendicular to the applied load), to the extension or axial strain (in the direction of the applied load).

“Shear modulus (G, S or μ)” is defined as the ratio of shear stress to the shear strain.

The present invention discloses a method of inactivating a virus, comprising sweeping a subject with a specific microwave frequency to induce a collective vibration of virus through microwave resonant absorption (MRA) without destructing a water and nucleic acid molecule. Such energy transfer is induced by the inherent core-shell charge structure of a virus through the dipolar mode of confined acoustic vibrations.

In the preferred embodiment of the present invention, the specific microwave frequency is a confined acoustic vibration (CAV) frequency, the MRA depends on the size and mechanical properties of a virus, the subject is an organism or an existing environment thereof.

The method is applied for general human or other organism with the illumination intensity below the IEEE recommended regulatory limit.

The present invention also discloses a device of inactivating a virus by using the virus inactivation method comprising (a) a microwave source; (b) a radiation unit, connecting with the microwave source, to radiate the microwave to the target.

In the device of the present invention, the microwave source is a YIG oscillator, or a combination of a YIG oscillator, a frequency multiplier, and a power amplifier. The radiation unit is an antenna or a radar. Then, the antenna is a horn antenna or a slot antenna.

The device of the present invention is a low power and frequency-tunable microwave and capable of being used for large area microwave virus inactivation.

### Examples

The examples below are non-limiting and are merely representative of various aspects and features of the present invention.

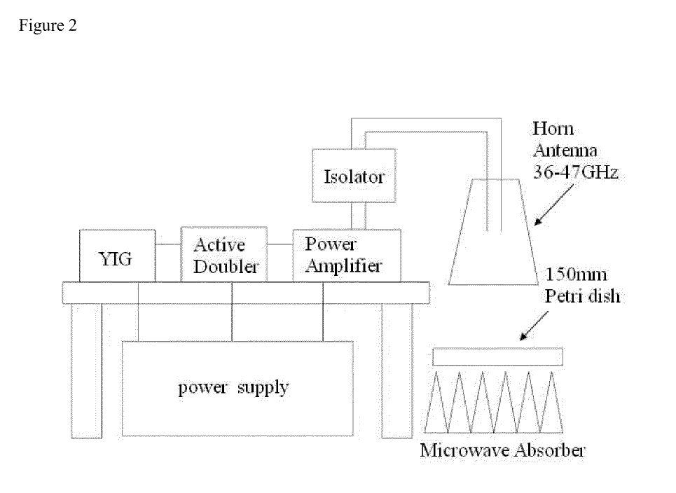

According to our pending U.S. patent Ser. No. (12/050,894, 2008 Mar. 18, Detect and identify virus by the microwave absorption spectroscopy) and previous results (T.-M. Liu et al. Microwave resonant absorption of viruses through dipolar coupling with confined acoustic vibrations. Appl. Phys. Lett. 94, 043902 (2009)), the presence of MRA has been validated. To investigate whether the microwave can further inactivate viruses at their MRA frequencies, we constructed a high-power and frequency-tunable microwave source (36-47 GHz) (See FIG. 2). The system employs a YIG oscillator (18-26 GHz) as a driving source. The frequency is doubled to 36-52 GHz by an active doubler. To increase the power level, we use a wide band amplifier (36-47 GHz) with 23 dBm P1 dB level. After passing through an isolator, the microwave is radiated by a horn antenna and incident on the petri dish containing the viruses. The illumination intensity is below 100 W/m², which is the IEEE recommend regulatory limit for general human exposure (R. K. Adair. Vibrational Resonances in Biological Systems at Microwave Frequencies. Biophys. J. 82, 1147-1152 (2002)). After 30 minutes of illumination, the viruses are diluted and their infectious titer is evaluated by an MTT assay.

MTT assay is a standard colorimetric assay to evaluate the activity of enzymes which can reduce the MTT ((3-(4,5-Dimethylthiazol-2-yl)-2,5-diphenyltetrazolium bromide, a tetrazole)) to purple formazan. By measuring the optical density (OD) of the insoluble purple formazan under different enzyme concentrations, the activity of enzyme can be semi-quantitatively evaluated. Because the mitochondrial dehydrogenase can reduce the MTT to formazan, MTT assay can thus be applied to indicate the cell viability in the test of cytopathic effects (CPE). To evaluate the infectious titer of viruses, the under test viral solution is sequentially diluted by 10 times. According to the OD values of totally survive and totally death, one can extract the dilution factors MTT₅₀/ml of 50% viability through interpolation. Then the infectious titer of MTT assay can be defined and calculated as −log₁₀(MTT₅₀/ml).

### Results

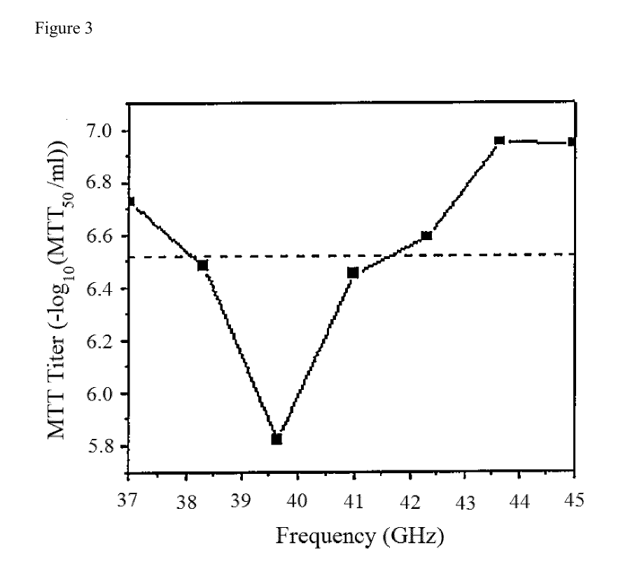

FIG. 3 is the microwave spectrum of MTT titer of EV71. There shows a resonant inactivation around 40 GHz, which is close to our previous measured MRA frequency of inactivated EV71. The shift of resonant frequency could be due to the size variation with strains, the inactivation processes, or the accuracy of microwave devices.

While the invention has been described and exemplified in sufficient detail for those skilled in this art to make and use it, various alternatives, modifications, and improvements should be apparent without departing from the spirits and scope of the invention.

One skilled in the art readily appreciates that the present invention is well adapted to carry out the objects and obtain the ends and advantages mentioned, as well as those inherent therein. The cells, viruses, and processes and methods for producing them are representative of preferred embodiments, are exemplary and not intended as limited on the scope of the invention. Modifications therein and other uses will occur to those skilled in the art. These modifications are encompassed within the spirit of the invention and are defined by the scope of the claims.

What is claimed is:

1. A non-thermal method of inactivating a virus, comprising sweeping a subject with a confined acoustic vibration (CAV) frequency to induce a collective vibration of a virus through microwave resonant absorption (MRA) without destructing a water and nucleic acid molecule.

2. The method according to claim 1, wherein the MRA is induced through the inherent core-shell charge structure of the virus through dipolar mode of CAV frequency.

3. The method according to claim 2, wherein the MRA depends on a size, and a mechanical property of the virus.

4. The method according to claim 3, wherein the mechanical property is selected from the group consisting of longitudinal sound velocity, transverse sound velocity, stiffness constant, bulk modulus, Poission' ratio, mass density, and shear modulus.

5. The method according to claim 1, wherein the subject is an organism or an existing environment thereof.

6. A device of inactivating a virus by using the method according to claim 1, comprising:
(a) a microwave source;
(b) a radiation unit, connecting with the microwave source, to radiate the microwave to the target.

7. The device according to claim 6, wherein the microwave source is a YIG oscillator, or a combination of a YIG oscillator, a frequency multiplier, and a power amplifier.

8. The device according to claim 6, wherein the radiation unit is an antenna or a radar.

9. The device according to claim 8, wherein the antenna is a horn antenna or a slot antenna.

10. The device according claim 6, which provides a low-power and frequency-tunable microwave.

11. The device according to claim 6, which is used for large area microwave virus inactivation.
